SEQUENCE LISTING (16 sequences)

SEQ ID NO: 1 - DNA, 18 bp, Artificial sequence
  Synthetic
ctatagtgagtcgtatta

SEQ ID NO: 2 - RNA, 31 nt, Artificial sequence
  Synthetic
gguguugacuguugaaucucauggcaacacc

SEQ ID NO: 3 - RNA, 39 nt, Artificial sequence
  Synthetic
gacugauguugacuguugaaucucauggcaacaccaguc

SEQ ID NO: 4 - RNA, 59 nt, Artificial sequence
  Synthetic
gagcuuaucagacugauguugacuguugaaucucauggcaacaccagucgaugggcugu

SEQ ID NO: 5 - RNA, 59 nt, Artificial sequence
  Synthetic
gagcuuaucagacugauguugacuuuugaaucucauggcaacaccagucgaugggcugu

SEQ ID NO: 6 - RNA, 59 nt, Artificial sequence
  Synthetic
gagcuuaucagacugauguugacuguugaaucucacggcaacaccagucgaugggcugu

SEQ ID NO: 7 - RNA, 59 nt, Artificial sequence
  Synthetic
gagcuuaucagacugauguacacuguugaaucucauggguacaccagucgaugggcugu

SEQ ID NO: 8 - RNA, 60 nt, Artificial sequence
  Synthetic
gagcuuaucagacugauguugacuguugaaucucauggucaacaccagucgaugggcugu

SEQ ID NO: 9 - RNA, 80 nt, Artificial sequence
  Synthetic
ggggaugagguaguagguuguauaguuuuagggucacacccaccacugggagauaacuauacaaucuacugucuuuccuc

SEQ ID NO: 10 - RNA, 37 nt, Artificial sequence
  Synthetic
gggaauuugugugguauccguauagucacagauuccc

SEQ ID NO: 11 - RNA, 39 nt, Artificial sequence
  Synthetic
gggagcugaaacacaguugguuuguguacacuggcuccc

SEQ ID NO: 12 - RNA, 40 nt, Artificial sequence
  Synthetic
gggaacuugugauguuuaccguuuaaauccacggguuccc

SEQ ID NO: 13 - RNA, 29 nt, Artificial sequence
  Synthetic
gagggguuuuugccuccaacugacuccuc

SEQ ID NO: 14 - RNA, 38 nt, Artificial sequence
  Synthetic
gggguagauuucuguguucguuaggcaacagcuacccc

SEQ ID NO: 15 - RNA, 29 nt, Artificial sequence
  Synthetic
ggcagaucugagccugggagcucucugcc

SEQ ID NO: 16 - RNA, 42 nt, Artificial sequence
  Synthetic
ggcaaaugaggcgcugcauguggcagucugccucucuuugcc

(19) United States
(12) Patent Application Publication
(10) Pub. No.: US 2023/0416247 A1
(43) Pub. Date: Dec. 28, 2023

(54) DRUG-LIKE MOLECULES AND METHODS FOR THE THERAPEUTIC TARGETING OF MICRORNA-21

(71) Applicant: University of Washington, Seattle, WA (US)

(72) Inventors: Matthew D. Shortridge, Seattle, WA (US); Gabriele Varani, Seattle, WA (US)

(73) Assignee: University of Washington, Seattle, WA (US)

(21) Appl. No.: 18/247,353

(22) PCT Filed: Sep. 28, 2021
(86) PCT No.: PCT/US2021/052382

§ 371 Date: Mar. 30, 2023

Related U.S. Application Data
(60) Provisional application No. 63/086,257, filed on Oct. 1, 2020.

(51) Int. Cl. C07D 471/04; C07D 401/12; A61K 45/06; A61P 35/00

(52) U.S. Cl. CPC C07D 471/04; C07D 401/12; A61K 45/06; A61P 35/00

(57) ABSTRACT

Compounds and compositions for inhibiting miR-21, methods for inhibiting miR-21 in a subject, and methods for treating a disease or condition treatable by decreasing the level of microRNA miR-21 in a subject.

### CROSS-REFERENCE TO RELATED APPLICATION

This application claims the benefit of U.S. Application No. 63/086,257, filed Oct. 1, 2020, expressly incorporated herein by reference in its entirety.

### STATEMENT OF GOVERNMENT LICENSE RIGHTS

This invention was made with government support under Grant Nos. R01 GM103834 and R35 GM126942, awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P1140WOUW_Seq_RAW-20210927.txt. The text file is 2 KB; was created on Sep. 27, 2021; and is being submitted via EFS-Web with the filing of the specification.

### BACKGROUND OF THE INVENTION

Natural products and fully synthetic antibiotics are well-known class of compounds which target ribosomal RNA (rRNA) and elicit a therapeutic response. Examples include aminoglycosides and macrolides which inhibit protein translation (FIG. 1) and many more examples. The successful targeting of rRNA suggests that targeting newly discovered non-coding RNAs (ncRNAs) with small drug-like molecules could provide new and novel therapeutic avenues to treating chronic and infectious diseases in humans, livestock and plants. While aminoglycosides and macrolides, as well as other natural products, bind bacterial rRNA, these compounds have limited specificity and bind many RNAs independently of sequence. Despite increased research efforts in both academic and industry settings, the identification of potent, specific and pharmaceutically attractive small molecules which bind to RNA potently and specifically remains a very significant challenge.

Similar to proteins, RNA sequences (e.g., mRNAs, tRNA, rRNA, riboswitches, ribozymes and more) can fold into elaborate three-dimensional structures that provide binding sites for small molecule ligands; ribosomal RNA and, especially, riboswitches provide excellent examples of recognition of structured RNAs by small molecules. However, the elaborate three-dimensional and higher order structures observed in riboswitches, RNA enzymes and ribosomal RNAs, are not common in non-coding RNAs (ncRNAs) and messenger RNAs (mRNAs), which are less structured and coated in the cell with single strand RNA binding proteins (ssRBPs) which keep the RNA unfolded. Simpler secondary structures, such as stem-loops, internal loops and bulges, are instead ubiquitous in ncRNAs and mRNAs, and well-known to perform regulatory functions, by providing binding sites for other RNAs (e.g., microRNAs, miRNAs), for RNA-binding proteins or by directly affecting access of the ribosome and of translational initiation factors during initiation of protein synthesis or during RNA localization and stability, and other steps of RNA biogenesis.

The RNA stem-loop or hairpin is the most common local secondary structure motif found in RNA and can form within the context of much larger sequences (mRNAs) or as discreet, stand-alone functional structures (e.g., in miRNA precursor species). Many regulatory functions are associated with RNA stem-loops in the healthy and diseased state of cells. In miRNA precursors, for example, the stem-loop provides binding sites for the processing enzymes Drosha (with its co-factors in the microprocessor complex) and Dicer (with its co-factor TRBP) which generate the mature functional form of the non-coding RNA, the mature miRNA of 20-22 nts. Within the 5′-UTRs of many mRNAs, for example in growth factors, housekeeping genes and many proto-oncogenes, formation of stable stem-loops near the 5′-end of the mRNA inhibits initiation of protein synthesis and reduces expression of the corresponding protein.

The generalized structure of an RNA hairpin is therefore a dsRNA helical region (the stem), with bulged or internal loop nucleotides interspersed within it, capped by an apical loop comprised of unpaired nucleotides, which can have varying length (FIGS. 2A-2C). However, specific targeting of these simple RNA secondary structures with drug-like molecules is believed to be very challenging because they are so superficially similar to each other and, it is often stated, devoid of distinctive binding pockets.

It has been shown that RNA hairpins can be targeted with very high affinity and specificity by using macrocyclic peptides. Arginine rich, macrocyclic peptides of 14 or 18 amino acids were synthesized to fold into stable anti-parallel beta-sheet hairpin structures, capped by a heterochiral D-Proline/L-Proline turn. Using this chemistry, rational, structure-based approaches ligands have been generated with low picomolar affinity and 10⁶-fold binding selectivity relative to closely related sequences and structures (FIGS. 2C and 5). However, these molecules penetrate cellular membranes and can target RNA hairpins inside cells, but they lack the more favorable pharmacologic properties generally associated with small drug-like molecules (delivery, localization, cell permeability, intracellular localization).

The dysregulation of miRNA processing plays an important role in many proliferative diseases, including cancer, fibrotic and cardiac indications, inflammation and sepsis, along with viral and bacterial infections. A classic example of a miRNA dysregulated in human disease is provided by the human microRNA miR-21, a well-known proto-oncogene, marker of fibrosis and a molecular link between inflammation and various chronic diseases, including irritable bowel disease (IBD). In multiple mouse models of different cancers, miR-21 plays a causal role in malignant transformation, metastatic spread and resistance to treatment; suggesting that pharmacological inhibition of this ‘oncomiR’ would reverse even late-stage cancer. Up-regulation of miR-21 is associated with many cancers and kidney fibrosis in patients, as well as IBD, while its knock down by genetic means or oligonucleotide analogues reduces disease progression. Under conditions where miR-21 is upregulated, the mature sequence post transcriptionally silences hundreds of genes that regulate multiple biological pathways, including multiple well-known tumor suppressors (e.g., PDCD4, PTEN and more), proto-oncogenes (CDK6, BCL2), metabolic pathways (PPara) tissue remodeling (RECk) and many others, inducing the development of diseased states through a variety of molecular mechanisms by acting on cells in a pleiotropic fashion.

Disruption of miRNA homeostasis leading to suppressed or increased expression can occur through both transcriptional and post-transcriptional mechanisms. The limiting step in generating functionally mature miRNAs is the canonical microRNA biogenesis cascade (FIG. 3), which involves both nuclear and cytoplasmic processing steps and active nucleocytoplasmic transport that reduce a long (>200 nts) primary miRNA (pri-miRNA) transcript to the mature 20-22 nts functional forms that executes gene silencing. The pri-miRNA transcript is first cleaved in the nucleus by the microprocessor complex, including proteins Drosha (the enzyme) and DGCR8 (an essential co-factor), generating a 60-70 nts precursor (pre-miRNA) stem loop. The pre-miRNA is exported to the cytoplasm by the exportin 5 factor and further processed by Dicer (the enzyme) and TRBP to release the functional mature miRNAs.

Both the pri- and pre-miRNA sequences fold to form a common stem-loop structure that is recognized by the enzymatic processing complexes (FIG. 3). Certain features are necessary for efficient processing, including a phosphorylated 5′-end and a 2-nt overhang at the 3′-end of the RNA, both of which are needed for proper orientation of the PAZ-domains of the enzymes on the RNA substrate. Dicer requires a helical stem comprised of roughly two helical turns capped by a large (about 10-15 nt) unstructured apical loop. However, local features recognized by specific domains of the processing proteins differ between different microRNA precursors. The variability in sequence and structure between pre-miRNAs suggests that each sequence could be selectively targeted in a structure- and sequence-dependent manner with small molecules.

The human microRNA miR-21 is generated by the canonical microRNA biogenesis pathway, starting from an independently transcribed primary transcript located on chromosome 17 (17q23.1) within the VPM1 protein-coding gene. The mature functional form of miR-21 is excised from within this much larger transcript and is highly conserved across vertebrates. The functionally mature miR21-5p sequence is located on the 5′-strand of the stem loop corresponding to its precursor, pre-miR-21, between residues U8 and A29 (FIG. 4A) as numbered from the primary sequence found in miRBASE v22. In many diseases, the 5p mature sequence is overexpressed and plays a pathogenic role. The mature miR-21 3′-sequence (miR-21-3p) has also been shown to be functionally important in multiple diseases, when over-expressed, but has been much more sparingly investigated.

Multiple efforts have sought the direct targeting of the mature fully processed miRNA sequences using anti-sense oligonucleotide chemistry and various oligonucleotide analogues. However, these programs have been unsuccessful. Therefore, a need exists for improved methods and compounds using small drug-like molecules, for blocking the biogenesis pathway that generates the pathogenic mature miR21-5p and miR-21-3p sequences, thereby reducing their over-expression in multiple pathogenic states. The present invention seeks to fulfill this need and provides further related advantages.

### SUMMARY OF THE INVENTION

The present invention provides compounds for inhibiting human microRNA miR-21 (miR-21), methods for inhibiting miR-21 in a subject, and methods for treating a disease or condition treatable by decreasing the level of miR-21 in a subject.

In one aspect, the invention provides compounds that bind to miR-21 and that are useful to disrupt the formation of RNA-protein complexes.

In certain embodiments, the invention provides a compound having formula (I):

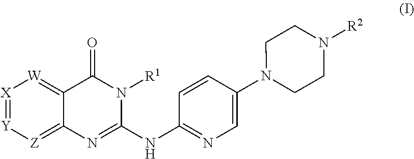

or a tautomer or pharmaceutically acceptable salt thereof, wherein:
W, X, Y, and Z are independently selected from
(a) N, or,
(b) CR³;
R¹ is selected from the group consisting of hydrogen and unsubstituted C1-C6 alkyl;
R² is selected from the group consisting of hydrogen and unsubstituted C1-C6 alkyl; and
R³ is selected from the group consisting of hydrogen and unsubstituted C1-C6 alkyl,
with the proviso that only one of W, X, Y, and Z is N; or W is CR³, X is N, and Y is CR³; or W, X, Y, and Z are CR³.

In certain embodiments, the compound has formula (II):

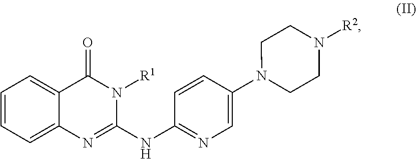

or a tautomer or pharmaceutically acceptable salt thereof.

In other embodiments, the compound has formula (III):

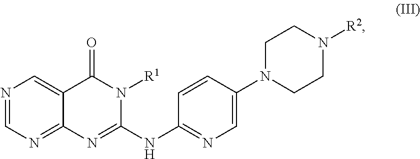

or a tautomer or pharmaceutically acceptable salt thereof.

In further embodiments, the compound has formulae (IVA), (IVB), (IVC), or (IVD):

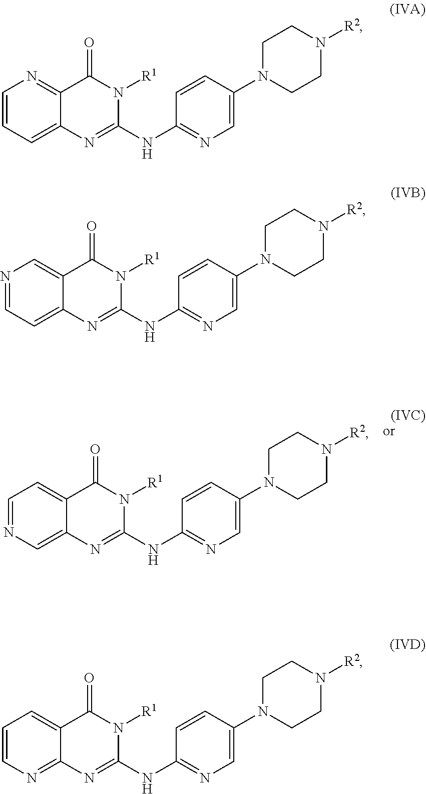

or a tautomer or pharmaceutically acceptable salt thereof.

In another aspect, the invention provides pharmaceutical compositions. In one embodiment, the pharmaceutical composition comprises a compound as described herein (e.g., a compound of formulae (I), (II), (III), (IVA), (IVB), (IVC), or (IVD)), or a tautomer or a pharmaceutically acceptable salt thereof, and a pharmaceutically acceptable carrier.

In further aspects, methods for using the compounds of the invention are provided.

In certain embodiments, the invention provides a method for decreasing the level of microRNA miR-21 in a cell.

In other embodiments, the invention provides a method for treating a disease or condition treatable by decreasing the level of microRNA miR-21 in a subject.

In certain embodiments of the methods described above, the invention further comprises administering to the subject a therapeutically effective amount of a second chemotherapeutic agent.

In further embodiments, the invention provides a method for inhibiting miR-21 in a subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is tabulation of RNA binding compounds.

FIGS. 2A-2C illustrate the general structural features of RNA stem-loop (hairpin) structures. FIG. 2A illustrates the secondary structure of the HIV-1 TAR RNA shows the common secondary structure elements found in RNA; including two double stranded stem regions, a three-nucleotide bulge and a six-nucleotide apical loop capping the helix. FIG. 2B illustrates the three-dimensional structure of the HIV-1 TAR RNA in the absence of any ligand (PDB 1 ARN) and FIG. 2C in the presence of the macrocyclic peptide ligand JB181 (PDB 6D2U). While the secondary structure remains the same regardless of whether a ligand is bound or not, the three-dimensional structure changes significantly. FIG. 2C illustrates how JB181 makes specific contacts to each stem region, the bulge and apical loop (PDB 6D2U). For RNA, the binding site of a ligand is not necessarily localized to a single site on a secondary structure element but can encompasses larger regions that fold around the ligand to generate unique binding pockets by induced fit.

FIG. 3 illustrates the canonical microRNA biogenesis pathway. The biogenesis pathway for most microRNAs includes both nuclear and cytoplasmic processing steps. In the nucleus, the primary transcript (pri-miRNA) generally transcribed by PolII is cleaved by the microprocessor (DROSHA/DGCR8) complex to form 59-72 nts RNA hairpin structures (pre-miRNAs). The pre-miRNA is exported to cytoplasm and further cleaved by the miRISC complex (DICER/TRBP/AGO2) to generate the sense (miRNA) and anti-sense (miRNA*) functional mature miRNA sequences.

FIGS. 4A and 4B illustrate that pre-miRNA stem-loops are characterized by large ‘open’ apical loop structures. FIG. 4A illustrates the secondary structure of the pre-miR21 stem-loop. The full-length sequence is shown and boxed; G8 and U65*** were swapped, compared to the wild-type sequence, to improve in vitro transcription yields for NMR analysis. The apical loop portion used in this report is boxed in lighter outline; A23 was switched to G23* to improve in vitro transcription yields. Dashes represent potential weak base pairs observed only at low temperature (5° C.). FIG. 4B illustrates the 1D ¹H imino region for both the short transcript (top) and the full length (bottom) pre-miR-21 show close correspondence of equivalent signals (black lines), indicating identical folding; both transcripts lack base NH signals from the apical loop region, which is unpaired and structurally dynamic at physiological temperatures.

FIG. 5 tabulates representative compounds of the invention.

FIG. 6 compares the binding of representative compounds of the invention (see FIG. 5) to pre-miR21. 1D ¹H NMR ligand-detect titrations of compounds, generated by titrating in increasing concentrations of RNA into a solution containing 100 μM of the relevant compound in buffer containing 50 mM deuterated Tris at pH 6.5, as well as 250 mM NaCl, 50 mM KCl and 2 mM MgCl₂. As increasing amounts of compounds are bound to the RNA in the course of the titration, the line width of the NMR signal increases, and the NMR peak height decreases. The fraction of compound bound can be calculated from the decrease in linewidth relative to an internal standard. Saturation of the curves to a value of 1 indicates the predominant presence of a single site with high binding affinity. Approximate binding constants can be calculated by fitting the data points to a binding isotherm. For reference, the data fit for compound −0052 corresponds to a K_{d}=200 nM, while compound −0045 corresponds to a K_{d}=600 nM.

FIGS. 7A-7D present NMR data showing that representative compounds of the invention bind to pre-miR-21 strongly (−0045 and −0052), ‘close’ the apical loop of pre-miR-21 and induce a specific conformation of pre-miR-21 that is processed inefficiently by Dicer-TRBP: Compounds 25 (or −0045) (FIGS. 7A and 7B) and 30 (or −0052) (FIGS. 7C and 7D are unique among pre-miR-21 binding compounds. They specifically close the apical loop structure of pre-miR-21, as demonstrated by the 1D 1H NMR spectra focused on the imino protons (FIGS. 7A and 7C), which reveal formation of new base pairs and closing of the apical loop. All data were collected at 800 Mhz and at 5° C. in buffer containing 50 mM deuterated Tris at pH 6.5 and 50 mM NaCl. For Compound 25 (FIG. 7A), a 1 mM sample of pre-miR-21 was titrated to 1.5 mM compound 25; new resonance signals for U31, G32, U43 and G44 demonstrate formation of the putative tandem UG/GU wobble pair in the apical loop when compound 25 was present. Compound 30 (FIG. 7C) induce a similar but stronger response under the same conditions, closing the U33:A42 base pair as well, in addition to the two GU pairs. Both compounds also select a unique conformational state of the pre-miR-21 hairpin as demonstrated by NOESY spectra recorded under the same conditions (FIGS. 7B and 7D); the characteristic position of the peak corresponding to a close contact between U27H3 and A47H2 corresponds to a specific conformation of pre-miR-21 that is processed inefficiently by Dicer-TRBP.

FIGS. 8A and 8B illustrate that representative compounds of the invention inhibit binding of DGCR8 to pre-miR-21, but not to Let-7: FIG. 8A shows formation of DGCR8-pre-miR-21 complex, as monitored in a gel shift assay, in the absence (square) or in the presence of increasing concentrations of Palbociclib (circles) and compound −0045 (triangles); FIG. 8A shows formation of DGCR8-pre-Let-7 complex, as monitored in a gel shift assay, in the absence (square) or in the presence of increasing concentrations of Palbociclib (circles) and compound −0045 (triangles).

FIGS. 9A and 9B compare Dicer assays: FIG. 9A is a gel image illustrating DICER/TRBP activity on pre-Let7 with (right) or without (left) 2 μM of compound 25; no significant difference in activity is observed in the presence or absence of compound 25; and FIG. 9B is a gel image illustrating pre-miR-21 processed with (right) or without (left) 2 μM compound 25; processing activity is visibly reduced by addition of compound 25.

FIGS. 10A-10F compare anti-proliferative activity of 10 nmoles of compounds −0045 and −0052 against gastric (AGS) (FIGS. 10B and 10C, respectively) and pancreatic (ASPC1) (FIGS. 10E and 10F, respectively) cancer cell lines, as measured as a function of time and compared to palbociclib (−000) (FIGS. 10A-AGS; and 10D-ASPC1) using standard MTS assays in the indicated cell lines; results were in triplicate with reported experimental uncertainty. Palbociclib and −0052 reduce the viability of both cell lines to a statistically significant extent (marked with asterisks) but −0045 does not.

FIGS. 11A-11F compare anti-proliferative activity of 5 nmoles of compounds −0045 and −0052 against gastric (AGS) (FIGS. 11B and 11C, respectively) and pancreatic (ASPC1) (FIGS. 11E and 11F, respectively) cancer cell lines, as measured as a function of time and compared to palbociclib (−000) (FIGS. 11A-AGS; and 11D-ASPC1), using standard MTS assays in the indicated cell lines; results were in triplicate with reported experimental uncertainty. Palbociclib and −0052 reduce the viability of both cell lines to a statistically significant extent (marked with asterisks) but −0045 does not.

FIGS. 12A and 12B compare levels of mature miR-21 in HeLa cells, as measured by standard qRT-PCR after 5 days of incubation in the presence of palbociclib and compounds −0045 (sm1), −0050 (sm3) and −0052 (sm5) (FIG. 12A); and levels of mature miR-21 in HeLa cells were measured by qRT-PCR after 4 days of incubation in the presence of palbociclib (a control) and compounds −0045 (sm1), −0050 (sm3) and −0052 (sm5) (FIG. 12B), normalized to miR-21 levels in the absence of any compound. Similar data were observed for cells grown under higher cell density conditions (10-fold higher initial concentration), which we believe are less reliable because cell density becomes too high after approximately 48 hrs. The two panels represent independent duplicates of the same experiment.

FIGS. 13A-13D compare levels of mature miR-21 and pre-mi-21 measured in gastric adenocarcinoma (AGS) (FIGS. 13A and 13B, respectively) and pancreatic cancer cell line (ASPC 1) (FIGS. 13C and 13D, respectively), normalized to internal controls (U6 or U8 snRNA), in the presence of 10 nmoles of palbociclib (used here as a negative control, not expected to affect microRNA levels) and compounds −0045 and −0052, 48 hours after incubation, relative to DMSO control, and measured by standard qRT-PCR. Statistically significant changes are labeled with asterisks.

FIGS. 14A-14D compare levels of mature miR-10b and pre-miR-10b measured in gastric adenocarcinoma (AGS) (FIGS. 14A and 14B, respectively) and pancreatic cancer cell line (ASPC 1) (FIGS. 14C and 14D, respectively), normalized to internal controls (U6 or U8 snRNA), in the presence of 10 nmoles of palbociclib (a negative control not expected to directly affect miRNA levels) and compounds −0045 and −0052, 48 hours after incubation, relative to DMSO control, and measured by standard qRT-PCR. We notice that levels of pre-miR-10b are very low in both cell lines, rendering the data very uncertain. Statistically significant changes are labeled with asterisks.

FIGS. 15A-15F illustrates that compound −0052 does not affect cellular levels of other miRNAs such as miR-16, miR-28, or miR-182. Levels of each microRNA were measured at different times post incubation (24-72 hours) by standard qRT-PCR, normalized to internal controls (U6 or U8 snRNA), in both gastric (AGC) and pancreatic (ASPC1) cell lines. Levels of the microRNA are generally the same, within experimental uncertainty, in the absence (DMSO) or presence of compound −0052 (30).

FIGS. 16A and 16B illustrate that compounds −0045 and −0052 increase levels of PDCD4 in AGS cells following 48 hours of incubation, relative to DMSO control and normalized to β-actin levels, as measured by Western blotting (FIG. 16A) with an antibody specific to PDCD4; 13-actin is provided as loading control. Data in FIG. 16B are reported as enhancement of PDCD4 levels, relative to DMSO control (no compound added).

FIG. 17 is a schematic illustration of the preparation of representative compounds of the invention.

FIG. 18 tabulates miR-21 sequences.

FIG. 19 tabulates RNA hairpins and miRNA hairpin sequences.

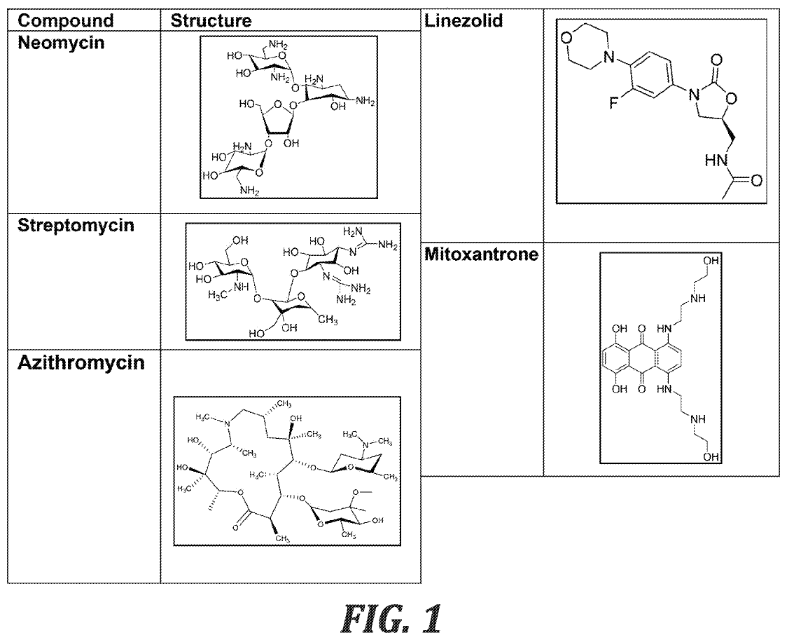

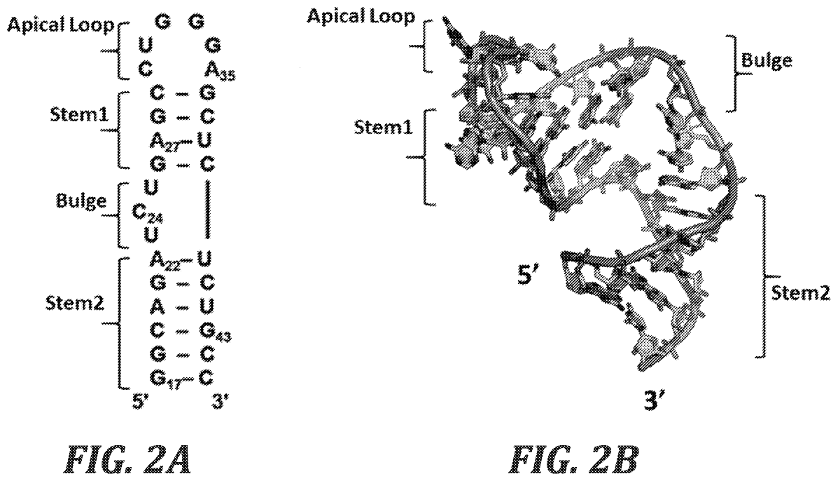

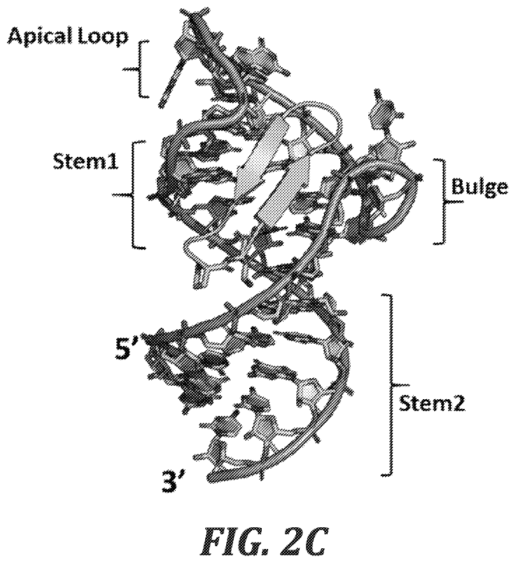

### DETAILED DESCRIPTION OF THE INVENTION

The present invention provides compounds for inhibiting miR-21, methods for inhibiting miR-21 in a subject, and methods for treating a disease or condition treatable by decreasing the level of microRNA miR-21 in a subject.

In one aspect, the invention provides compounds useful for inhibiting miR-21 in a subject and method for treating a disease or condition treatable by decreasing the level of microRNA miR-21 in a subject. The compounds bind to structured microRNA precursor miR-21 and are useful to reduce expression of the mature functional forms of the non-coding RNA miR-21.

In certain embodiments, the invention provides a compound having formula (I):

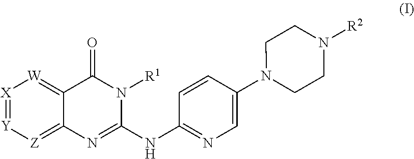

or a tautomer or pharmaceutically acceptable salt thereof, wherein:
W, X, Y, and Z are independently selected from
(a) N, or,
(b) CR³;
R¹ is selected from the group consisting of hydrogen and unsubstituted C1-C6 alkyl;
R² is selected from the group consisting of hydrogen and unsubstituted C1-C6 alkyl; and
R³ is selected from the group consisting of hydrogen and unsubstituted C1-C6 alkyl,
with the proviso that only one of W, X, Y, and Z is N; or W is CR³, X is N, and Y is CR³; or W, X, Y, and Z are CR³.

As used herein, the term “unsubstituted C1-C6 alkyl” refers to an unsubstituted straight chain and branched C1-C6 alkyl group (e.g., methyl, ethyl, n-propyl, i-propyl, n-butyl, s-butyl, t-butyl, n-pentyl, i-pentyl, s-pentyl, neopentyl, n-hexyl, i-hexyl, s-hexyl groups.)

In certain embodiments, the compound has formula (II):

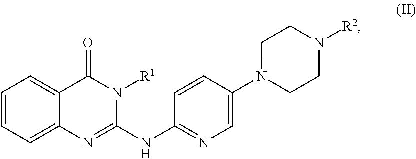

or a tautomer, or pharmaceutically acceptable salt thereof. In certain of these embodiments, R¹ is hydrogen and R² is hydrogen (e.g., compound 24). In other of these embodiments, R¹ is methyl and R² is hydrogen (e.g., compound 26).

In other embodiments, the compound has formula (III):

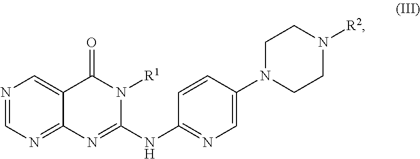

or a tautomer, or pharmaceutically acceptable salt thereof. In certain of these embodiments, R¹ is hydrogen and R² is hydrogen (e.g., compound 29).

In further embodiments, the compound has formulae (IVA), (IVB), (IVC), or (IVD):

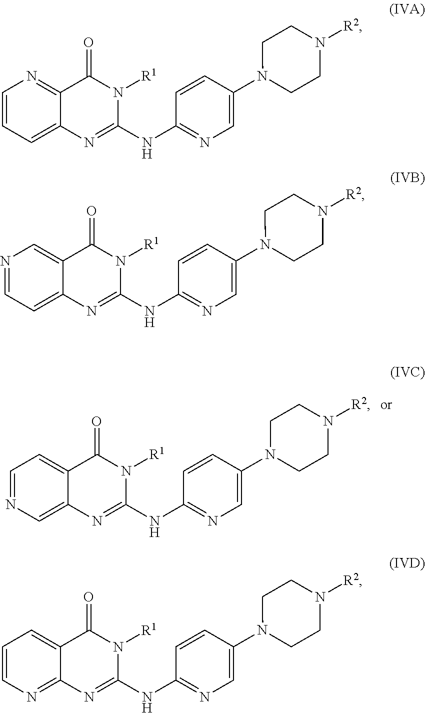

or a tautomer, or pharmaceutically acceptable salt thereof. In certain of these embodiments, the compound has formula (IVA), wherein R¹ is hydrogen and R² is hydrogen (e.g., compound 25). In other of these embodiments, the compound has formula (IVB), wherein R¹ is hydrogen and R² is hydrogen (e.g., compound 28). In further of these embodiments, the compound has formula (IVC), wherein R¹ is hydrogen and R² is hydrogen (e.g., compound 30). In another of these embodiments, the compound has formula (IVD), wherein R¹ is hydrogen and R² is hydrogen (e.g., compound 27).

In another aspect, the invention provides pharmaceutical compositions. In one embodiment, the pharmaceutical composition comprises a compound as described herein (e.g., a compound of formulae (I), (II), (III), (IVA), (IVB), (IVC), or (IVD)), or a tautomer or a pharmaceutically acceptable salt thereof, and a pharmaceutically acceptable carrier.

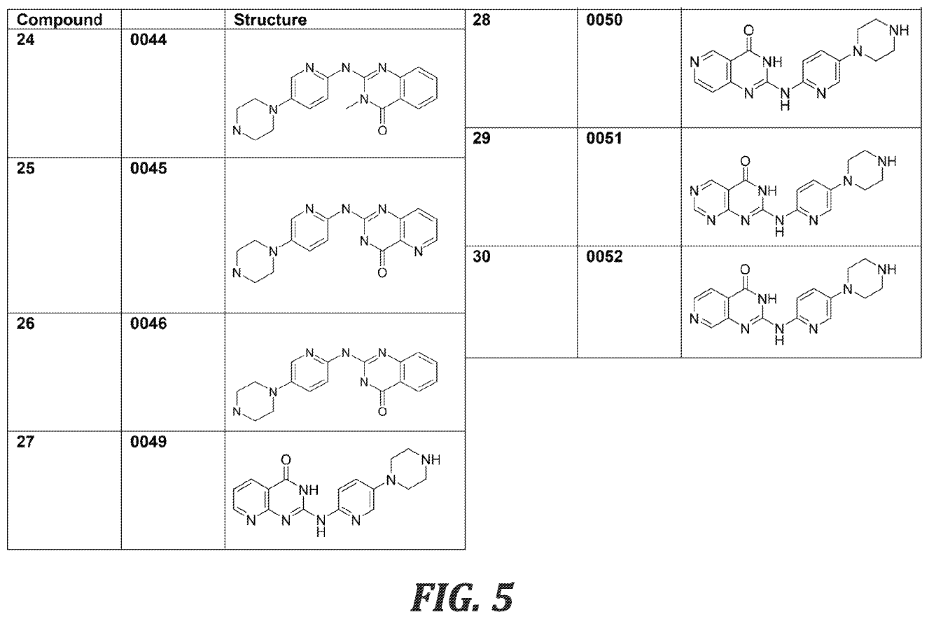

A representative subset of compounds is presented in FIG. 5, or tautomers or pharmaceutically acceptable salts thereof.

In another aspect, the invention provides pharmaceutical compositions. In one embodiment, the pharmaceutical composition comprises a compound as described herein (e.g., a compound of formulae (I), (II), (III), (IVA), (IVB), (IVC), or (IVD)), or a pharmaceutically acceptable salt thereof, and a pharmaceutically acceptable carrier.

In further aspect, methods for using the compounds are provided.

In certain embodiments, the invention provides a method for decreasing the level of microRNA miR-21 in a cell. In one embodiment, the method comprises contacting a cell having upregulated miR-21 with an amount of a compound as described herein (e.g., a compound of formulae (I), (II), (III), (IVA), (IVB), (IVC), or (IVD)), or a tautomer, or pharmaceutically acceptable salt thereof, effective to decrease the level of microRNA miR-21 in the cell. In certain of these embodiments, decreasing the level of microRNA miR-21 in the cell comprises binding a compound as described herein, or a tautomer, or pharmaceutically acceptable salt thereof, directly to primary miRNA transcripts or directly to pre-miRNA transcripts.

In other embodiments, the invention provides a method for treating a disease or condition treatable by decreasing the level of microRNA miR-21 in a subject. In one embodiment, the method comprises administering to a subject in need thereof a therapeutically effective amount of a compound as described herein (e.g., a compound of formulae (I), (II), (III), (IVA), (IVB), (IVC), or (IVD)), or a tautomer, or pharmaceutically acceptable salt thereof. In certain of these embodiments, the disease or condition is proliferative disease or condition, irritable bowel disease (IBD), fibrosis of the kidney or liver, or other degenerative liver conditions (e.g., NASH). In certain embodiments, the disease or condition is a cancer. Representative cancers treatable by decreasing the level of microRNA miR-21 include glioma, glioblastoma, breast, ovarian, cervical, colon, non-small cell lung cancer, lung adenocarcinomas, gastric, pancreas, prostate, stomach, colorectal, liver, hepatoma and hepatocellular carcinomas, oral and tongue squamous cell carcinomas, head and neck, and larynx cancers. In other embodiments, the disease or condition is proliferative disease or condition is a hematological malignancy. Representative hematological malignancies include myelomas and lymphomas including B-cell lymphoma, chronic lymphocyte lymphoma, T-cell acute lymphoblastic lymphoma, Richter syndrome, and leukemia.

In certain embodiments of the methods described above, the invention further comprises administering to the subject a therapeutically effective amount of a second chemotherapeutic agent. Suitable second chemotherapeutic agents include therapeutic agents know to be effective in the treatment of the specific cancer.

In further embodiments, the invention provides a method for inhibiting miR-21 in a subject. In one embodiment, the method comprises administering to a subject in need thereof an amount of a compound as described herein (e.g., a compound of formulae (I), (II), (III), (IVA), (IVB), (IVC), or (IVD)), or a tautomer, or pharmaceutically acceptable salt thereof, effective to inhibit miR-21 in the subject.

In certain embodiments of the methods described herein, the subject is a human.

As noted above, the direct targeting of the mature fully processed miRNA sequences using anti-sense oligonucleotide chemistry and various oligonucleotide analogues has been reported. However, in the practice of the invention, the compounds described herein block the biogenesis pathway by which these two mature sequences (mature miR21-5p sequence and mature miR-21-3p sequence) are generated in the cell. The compounds described herein are small drug-like molecules and are not oligonucleotide analogues.

As described herein, the invention provides compounds that suppress the activity of pathological microRNAs by directly binding to primary and pre-miRNA transcripts, thereby selectively inhibiting endonucleolytic processing by the Dicer and Drosha enzymes, thereby resulting in altered expression of a specific microRNA.

The small molecule compounds described herein target a specific local structure at the junction between the apical loop and helical stem of many pre-miRNAs, which is required for efficient processing and is found in a subset of microRNAs, including miR-21 and the pro-metastatic miR-10b, as well as other microRNAs. Reducing the levels of miR-21 to normal levels with small molecules targeting miRNA biogenesis, is expected to have therapeutic benefits in a variety of cancers and fibrotic and inflammatory conditions, for example liver and kidney fibrosis and irritable bowel disease. Thus, compounds described herein achieve the same cellular response as oligonucleotides in suppressing the activity of disease-associated microRNAs but avoid the well-known pharmacological pitfalls of oligonucleotide chemistry (low physiological delivery; non-specific organ distribution; reticuloendothelial system clearing and endosomal trafficking) by residing in the conventional favorable ‘Lipinski’ drug-like chemical space.

As noted above, the pre-miR-21 sequence is a 59 nt hairpin structure generated by Drosha cleavage at positions U7 and U66 (numbers are per miRbase) of the pri-miRNA. This structure is present in the pri-miRNA-21 as well and is recognized by Drosha-DGCR8 to execute the nuclear processing reaction, together with features outside of this stem-loop. Once excised, the pre-miRNA is recognized by Dicer-TRBP in the cytoplasm to execute the cytoplasmic processing reactions.

microRNA Processing

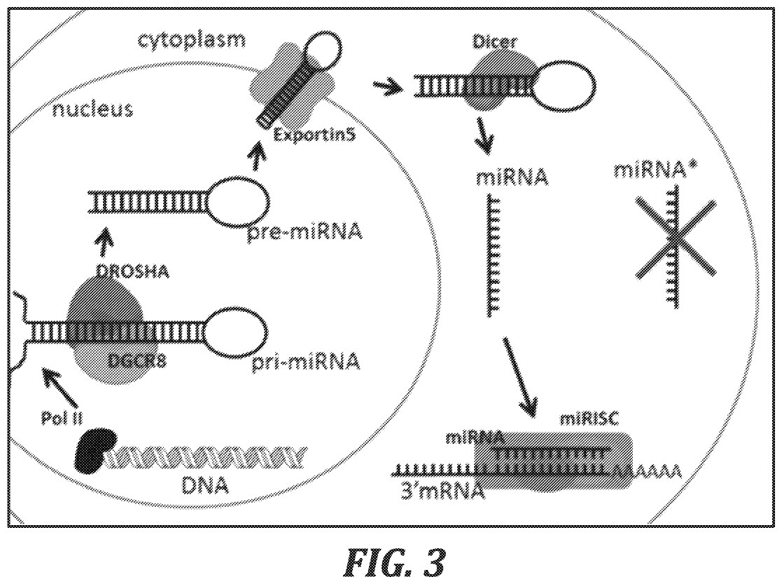

The canonical micoRNA biogenesis pathway is schematically illustrated in FIG. 3 and involves both nuclear and cytoplasmic processing steps to generate the functionally mature 22 nt single stranded miRNA.

Cytoplasmic processing of pre-miRNAs is carried out by the miRNA Induced Silencing Complex (miRSC). The miRISC recognizes both the apical loop and the 3′-end of the RNA to locate and orient the enzyme on the substrate to generate the mature 22 nt long single stranded sequence. The minimal human miRISC loading complex is composed of the RNA binding proteins (RBPs), Dicer, TAR RNA Binding Protein (TRBP), and Argonaute 2 (Ago2). The endoribonucleolytic enzyme Dicer is an RNAseIII multi-domain 218 kDa protein which cleaves both dsRNA and pre-miRNA hairpins efficiently but non-specifically. The RNA binding protein, TRBP contains three dsRNA binding domains and improves Dicer processing of pre-miRNAs relative to dsRNA fragments, thereby imparting some specificity in the cell for pre-miRNA substrates; Ago2 is a multifunctional protein, which has catalytic activity as well, but does not play a direct role in generating the mature miRNA sequence. Rather, the Ago2 protein participates in loading mature miRNAs onto target mRNAs following processing by Dicer-TRBP.

The present invention is focused on inhibiting the generation of the mature miRNA sequence: the activity of the complex of Dicer and TRBP that executes endonucleolytic cleavage in the cytoplasm, as well of Drosha-DGCR7 that generates the precursor pre-miRNA in the nucleus (FIG. 3).

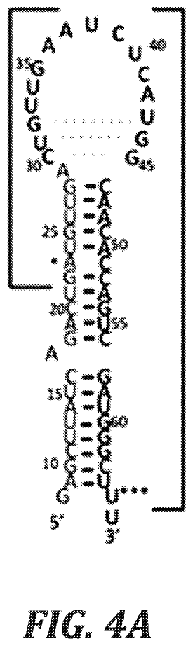

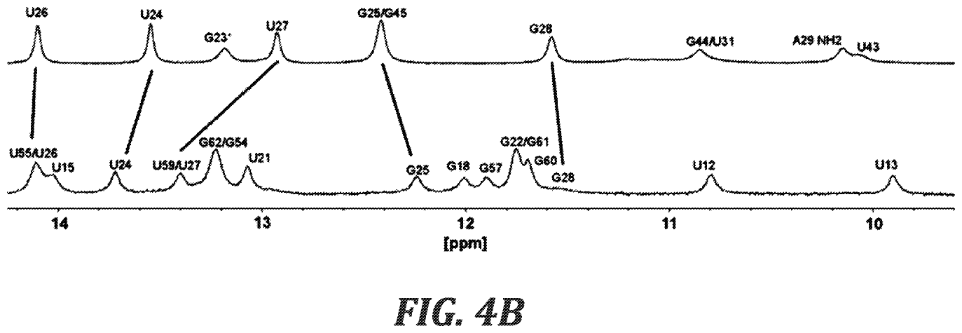

A ‘short’ RNA hairpin sequence as been used in the past as a model to study pre-miR-21. Such as short construct is shown in FIG. 4A and encompasses nucleotides G22-C52. The three-dimensional structure of this short hairpin by itself and in complex with a small cyclic peptide (PDB 5UTZ and 5UZZ) has been reported. That work was unable to identify the exchangeable proton resonances for any of the remaining loop nucleotides (U31-G44), demonstrating the absence of any stable base pair across the loop, and observed reduced NOE intensity for many non-exchangeable protons, consistent with a highly dynamic structure. These observations conclusively support the lack of stable structure in the apical loop. However, only the full-length is the substrate recognized by Dicer. In both the short pre-miR-21 and the full length pre-miR-21 sequences (FIG. 4B), exchangeable proton signals were not observed for the putative tandem U31:G44/G32:U43 wobble pairs predicted by computational folding; either the base pairs do not form at all or are only formed transiently and are unstable. This observation is relevant to the biochemical mechanism of action of the compound reported in this application.

miR-21 Binding Compounds

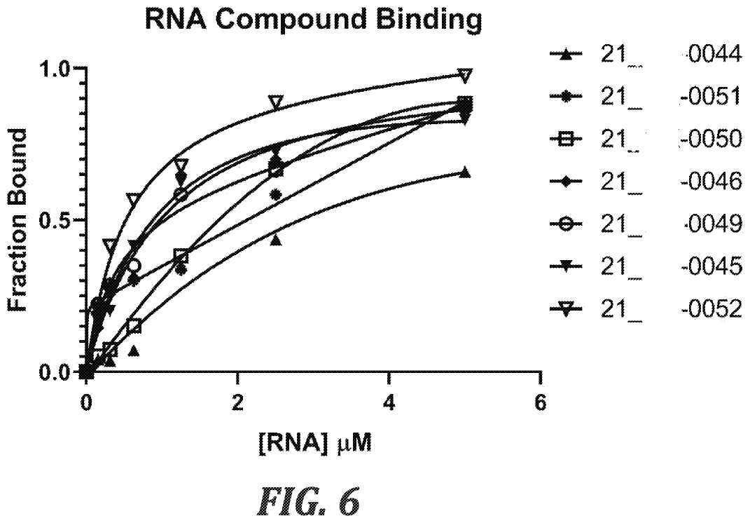

Representative compounds of formula (I) described herein are shown in FIG. 5. The compounds show a strong pre-miR21 binding capacity. For compound 25 (0045), the K_{D} was estimated to be 0.6 μM, while compound 30 (0052) had a K_{D} of 0.2 μM (FIG. 6); other compounds in the series have lower K_{d} in the high nM or low μM range.

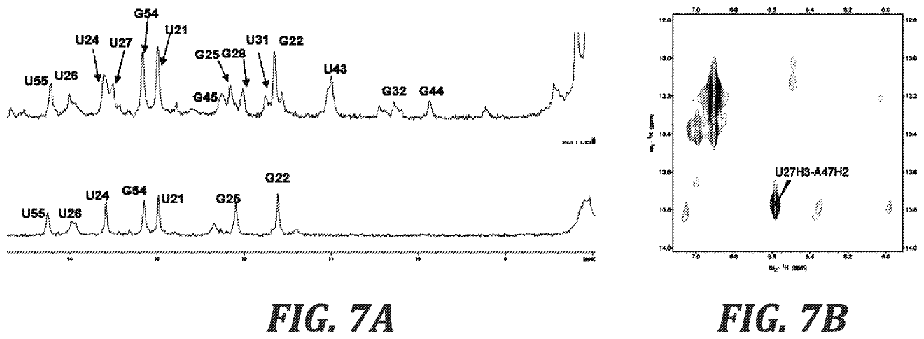

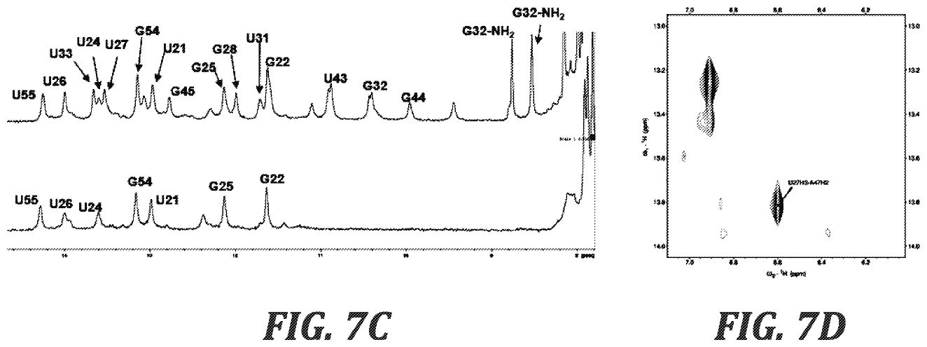

Remarkably, the compounds have the unique capacity to ‘close the loop’ of pre-miR-21. This large change in RNA structure is shown in FIGS. 7A-7D, compared to the unbound RNA which are shown at the bottom of FIGS. 7A and 7C. Titration of pre-miR-21 with compounds 25 (−0045) and 30 (−0052) very clearly show the emergence of new imino NH chemical shift resonances for the apical loop portion of the structure that have not been observed before or ever reported in the scientific literature. The effect is very similar to what is observed with mutations that stabilize the loop structure by inducing stable formations of base pairs in place of unstable base pairs, and that also reduce miR-21 expression in both biochemical experiments and in cells, as reported in the literature. Accordingly, the compounds of formula (I) are specific pre-miR-21 binding compounds with the capacity to inhibit enzymatic processing of this oncogenic miRNA sequence. The biochemical and cellular data are set forth below.

Biochemical Activity of miR-21 Binding Compounds

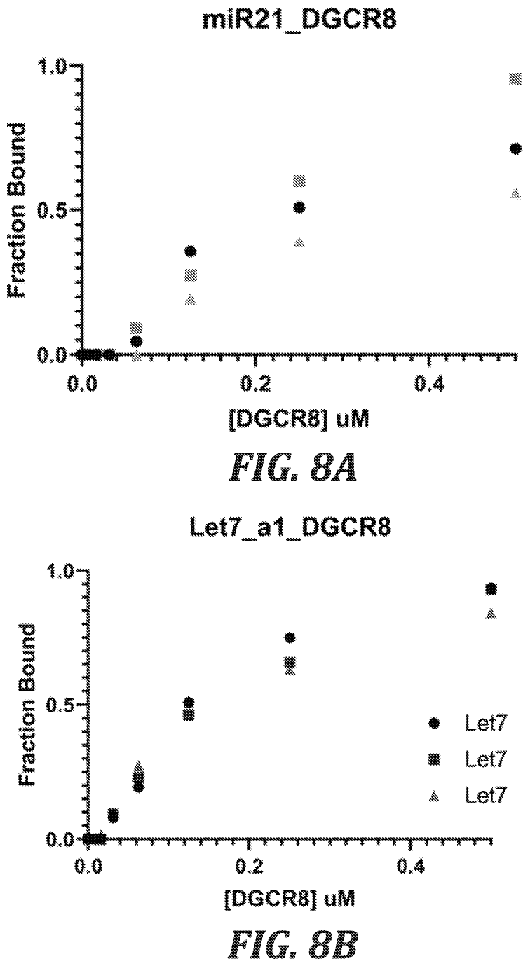

The ancillary micro-processor protein component DGCR8 is needed for accurate and efficient DROSHA cleavage of the pri-miRNA transcripts. DGCR8 binds the apical loop of pri-miRNA hairpins. Representative compounds of formula (I) bind to pri-miR-21 and induce specific changes in structure (FIGS. 7A-7D) which inhibit binding of this critical protein, which requires unpaired nucleotides, that form instead base pairs in the presence of compounds −0045 and −0052. Without DGCR8 binding to the apical loop, pri-miRNA processing is significantly inhibited, and generation of non-sense sequences is reported. To execute this inhibition assay, two DGCR8 constructs, amino acids 285-710 or 285-773, were purified (amino acids included in the longer construct are required for interaction with DROSHA); in both cases, we observed inhibition of binding of DGCR8 to pre-miR-21, but not to effect is observed on pre-Let-7 (FIGS. 8A and 8B), which lacks the motif efficiently recognized by DGCR8.

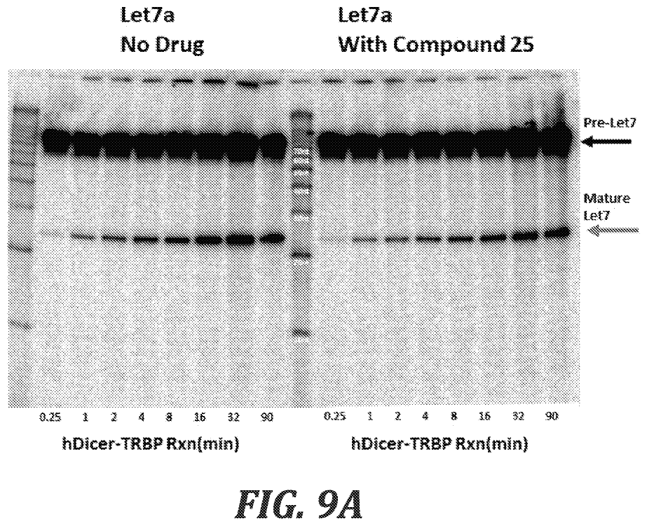

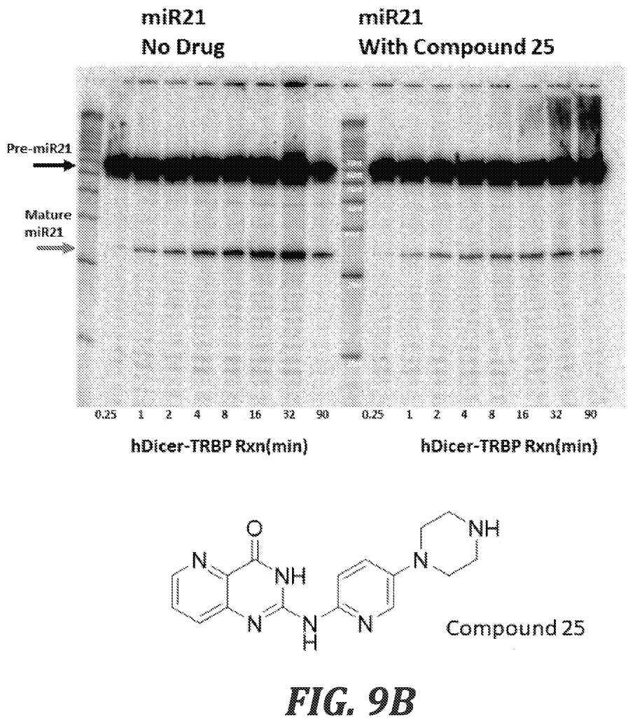

Compounds 25 and 30 (−0045 and −0052, respectively) specifically inhibit Dicer processing as well. In order to further assess the biochemical activity of our compounds, processing of pre-miR-21 and pre-Let7 by Dicer-TRBP in vitro was compared. FIGS. 9A and 9B show selective response in the rate of processing under early phase reaction conditions, before significant product builds up, for pre-miR21 compared to pre-Let7. When DICER/TRBP activity was tested on pre-Let7 with or without 2 uM of compound 25, no significant difference in activity is observed in the presence or absence of compound 25. In contrast, compound 25 visibly reduced pre-miR-21 processing by Dicer-TRBP, relative to control (no compound).

The results are specific to the initial rates (0.25-5 min) of the reaction. After the first 5-10 min of the reaction, the enzymatic assays exhibit product inhibition in addition to small molecule inhibition. This is inevitable because Dicer binds the product tightly. This assay and method of inhibiting miR-21 synthesis is different than typical enzymatic assays. First, the small molecule is targeting the substrate of the enzymatic assay. High affinity compounds that change the structure of the substrate may reduce processing but might not inhibit processing completely. Rather, changes in the specificity constant (kcat/Km), are a better measure of small molecule inhibition than traditional Ki's.

Cellular Activity

Three sets of assays were conducted in various cell lines to assess the activity of our compounds aimed at measuring cellular phenotypic response (proliferation rates and cellular morphology); levels of mature and pre-miR-21; levels of the well-known downstream target of miR-21, PDCD4.

Phenotypic Response: Proliferation of Transformed Cell Lines

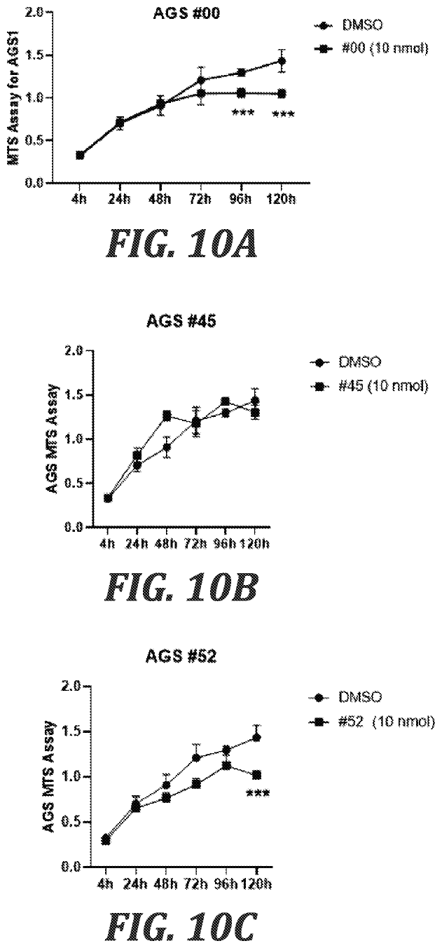

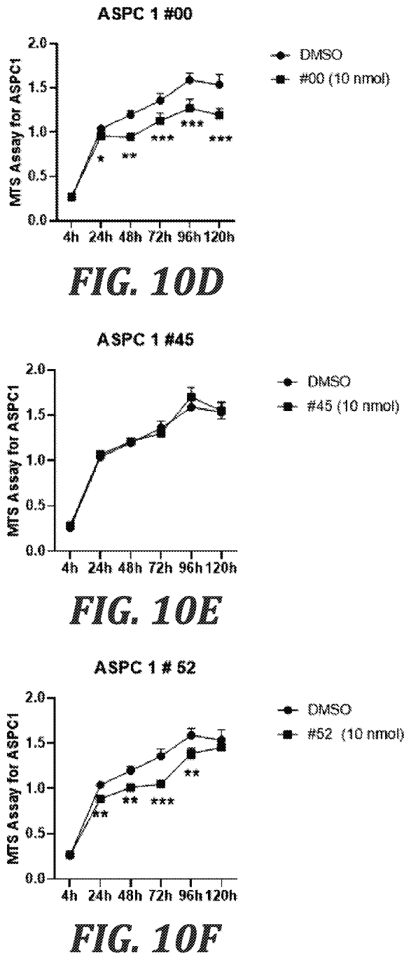

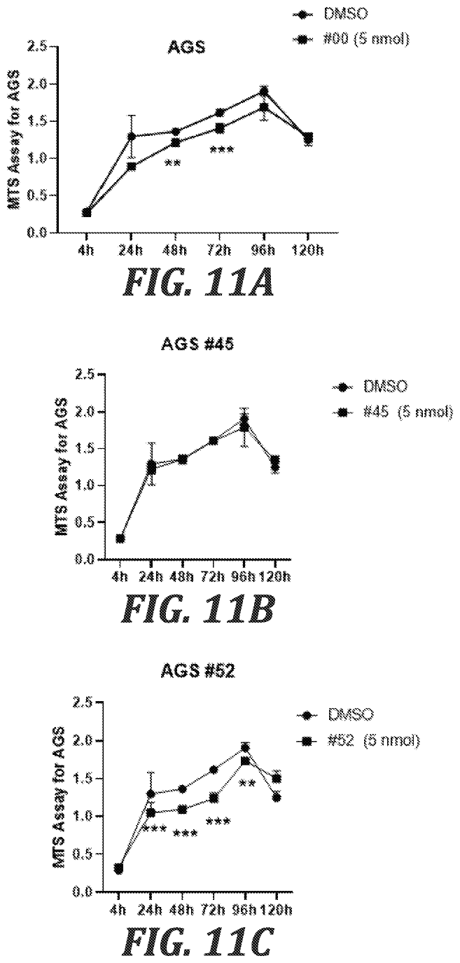

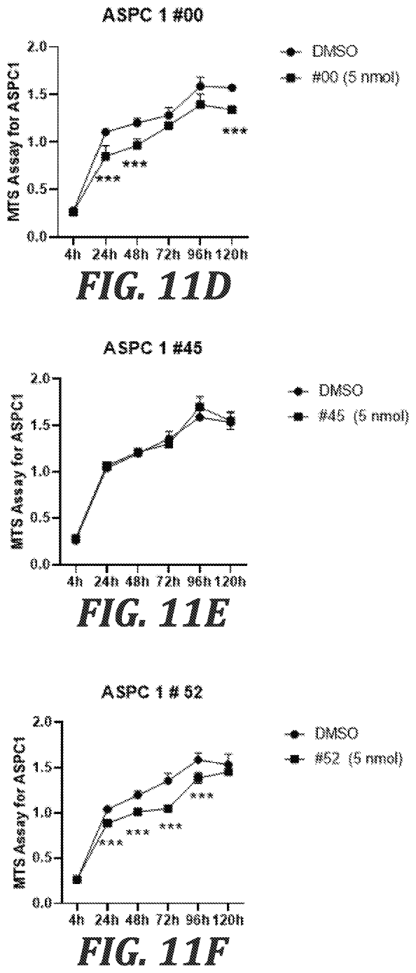

The anti-proliferative activity of compounds −0045 and −0052 in two cell lines characterized by high levels of miR-21 (gastric adenocarcinoma (AGS) and pancreatic (ASPC1) cancer origin) was determined. These experiments were conducted in a double-blind format, without knowledge of the identity or binding activity of the compounds, to avoid any unintentional bias. Cell killing activity for palbociclib, a positive control because it is a well-known cancer drug, as well as compound −0052, at both concentrations tested (10 and 5 nmols of compound) was observed (but not for compound −0045) in standard MTS assays conducted over 6 days (FIGS. 10A-10F and FIGS. 11A-11F display data obtained at the lower concentration of compounds). Viability in the presence of both palbociclib and compound −0052 is decreased after approximately 72-96 hrs to a statistically significant extent (data points marked with asterisks are statistically significant).

Levels of Mature miR-21 and its Downstream Target PDCD4 in the Presence of Representative Compounds

Compounds −045 and −0052 were shown to affect miR-21 and PDCD4 levels in multiple cell lines.

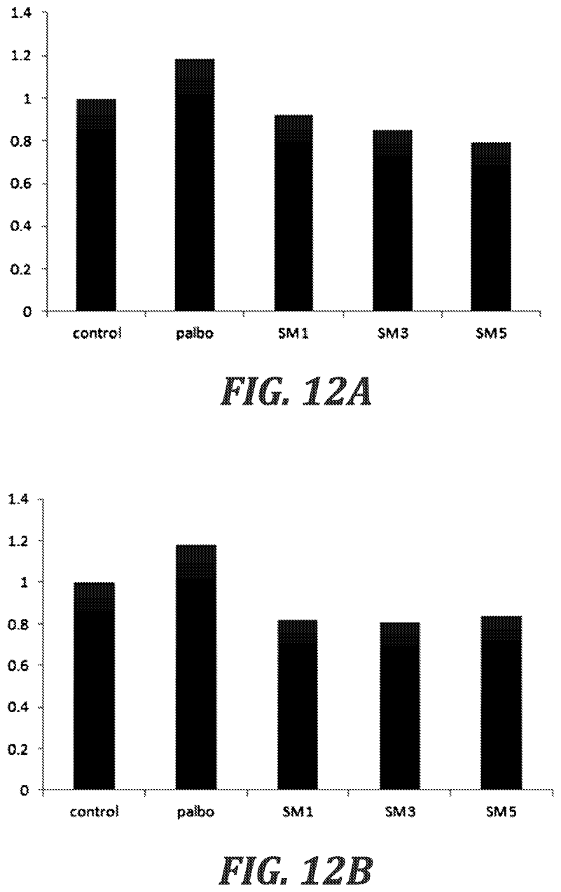

HeLa cells have relatively high levels of miR-21, and this cell line was used to evaluate whether addition of the molecules would decrease levels of mature miR-21, as expected for inhibition of miR-21 processing. In multiple experiments, with different cell densities (20 k vs 200 k cells/plate at the start of the experiment), at both days 4 and 5 of incubation, a consistent 10-20% increase was observed in levels of miR-21 induced by Palbociclib, which was used as a negative control of an anti-proliferative compound not expected to affect miR-21 levels, induced a small but consistently observed increase. In contrast, compounds −0045, −50 and −52, induce an approximately 20% percent decline in levels of mature miR-21 (FIGS. 12A and 12B; the comparison of FIG. 12A and FIG. 12B provides an indication in the uncertainty of the data). miR-21 is very long lived, with a lifetime of approximately 5 days. Thus, if synthesis of all new miR-21 was completely inhibited, a decrease of mature miR-21 levels to about 35% would be expected; thus, the 20% decrease observed corresponds to a reduction of about 60% of all newly synthesized miR-21 in this cell line. Similar results were obtained at higher numbers of cells/plate (200 k vs at the start of the experiment), but the experiment could not be extended beyond 2 days because of the very high cell density reached. The observed trends were nonetheless in agreement with the experiments conducted at lower cell density.

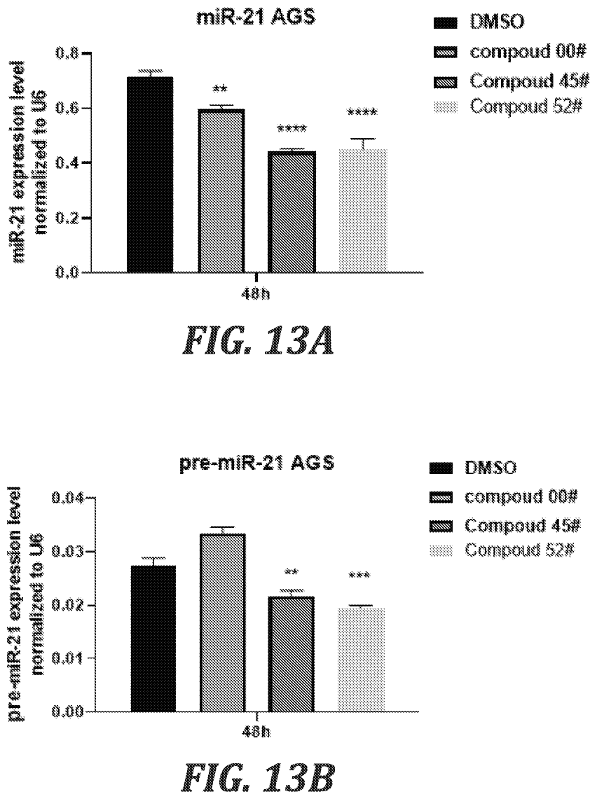

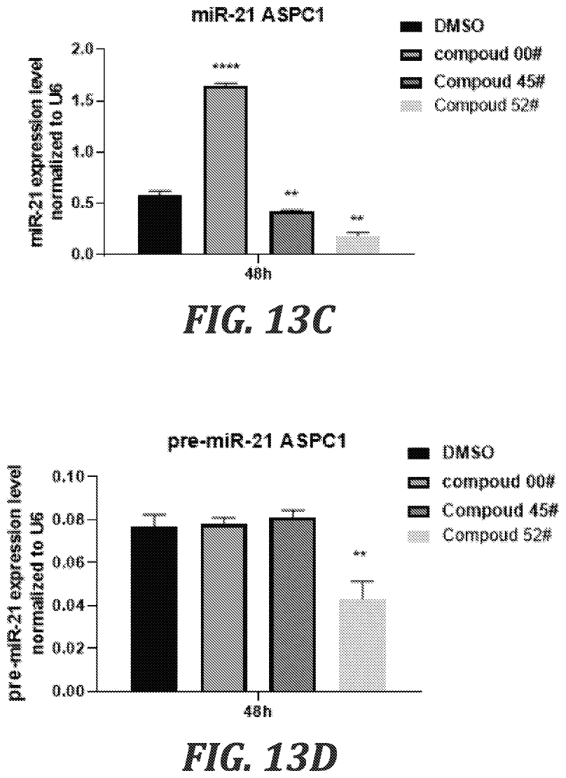

Levels of mature miR-21 and its precursor pre-miR-21 were measured in gastric carcinoma (ASG) and pancreatic (ASPC1) cancer cell lines under conditions where we observed a decrease in proliferation (FIGS. 13A-13D), using the same blind format described above. Significant (40%) decreases were observed in mature miR-21 levels in both AGS and ASPC1 cells challenged with both −0045 and −0052, and less significant changes with the control palbociclib, a kinase inhibitor not expected to reduce miR-21 levels (FIGS. 13A-13D). Reductions in mature miR-21 were also accompanied by a reduction in pre-miR-21 levels, suggesting that at least part of the inhibition occurs prior to Dicer processing, although measurements of the pri-miR-21 levels are required to confirm this conclusion.

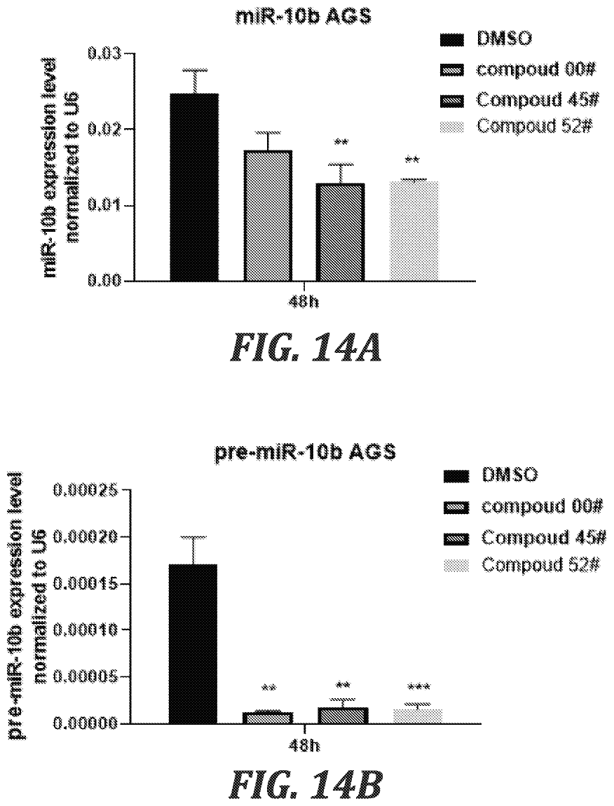

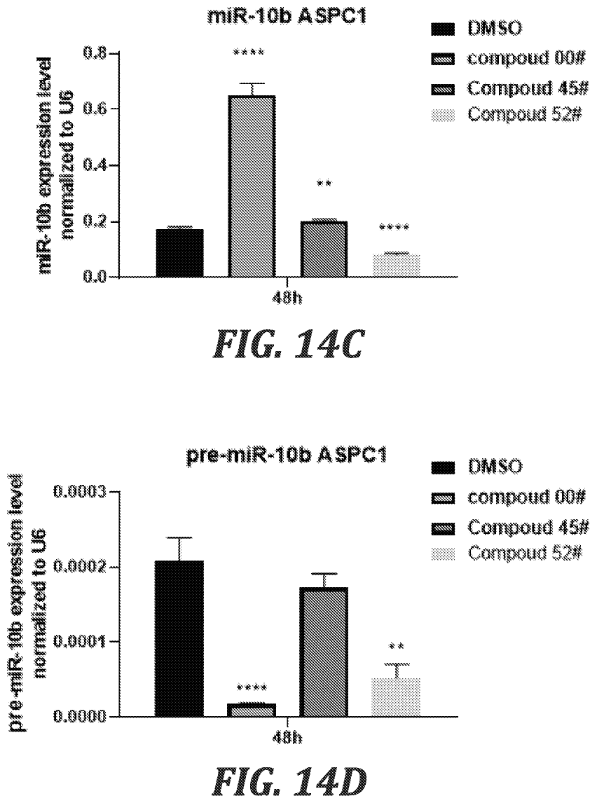

In order to investigate whether inhibition was specific to miR-21, levels of mature miR-10b and pre-miR-10b were examined in the same cell lines (FIGS. 14A-14D); this is another oncogenic microRNA implicated in metastasis. Compound −0052 reduces levels of mature miR-10b in both AGS and ASPC1 cells (about 2-fold), while compound −0045 displays smaller and not statistically significant changes in ASPC1 cells. Strong suppression of pre-miR-10b levels in AGS cells, and smaller decreases in ASPC1, were observed consistent with the changes in the mature miR-10b levels, but levels of pre-miR-21 are very low and experiments are therefore noisy.

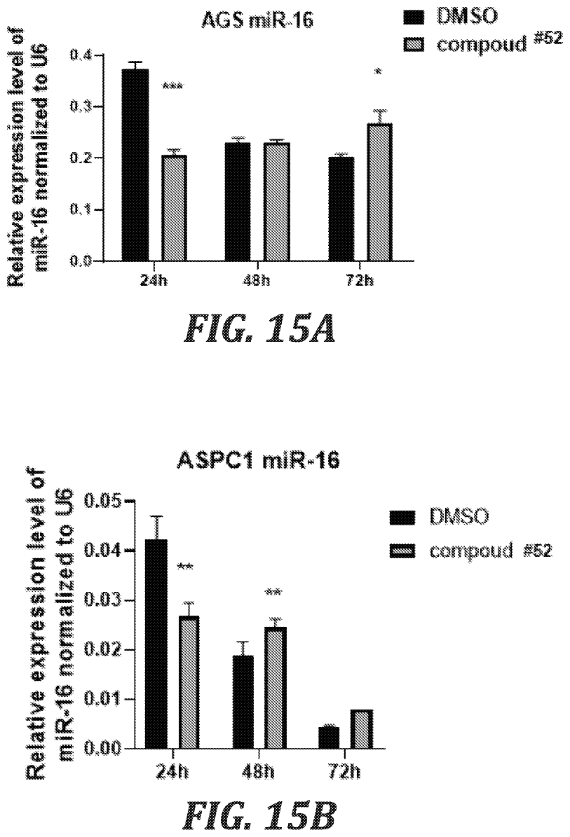

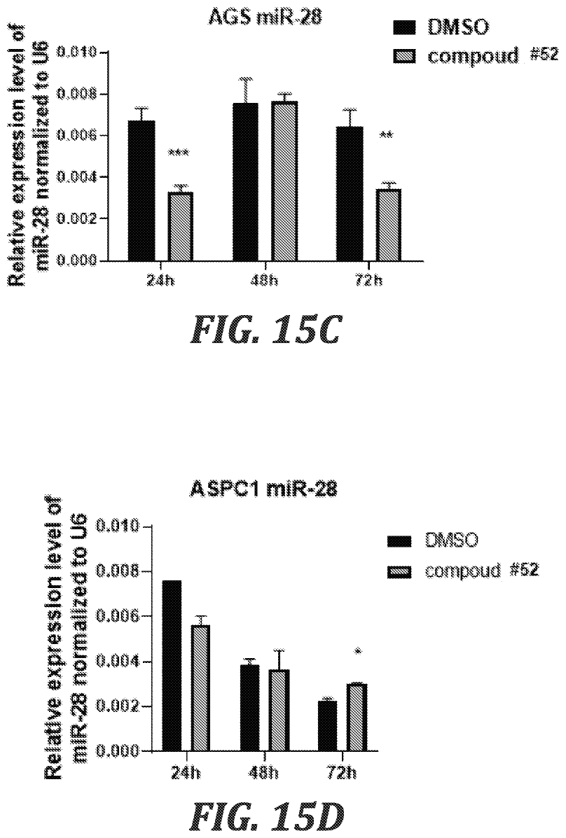

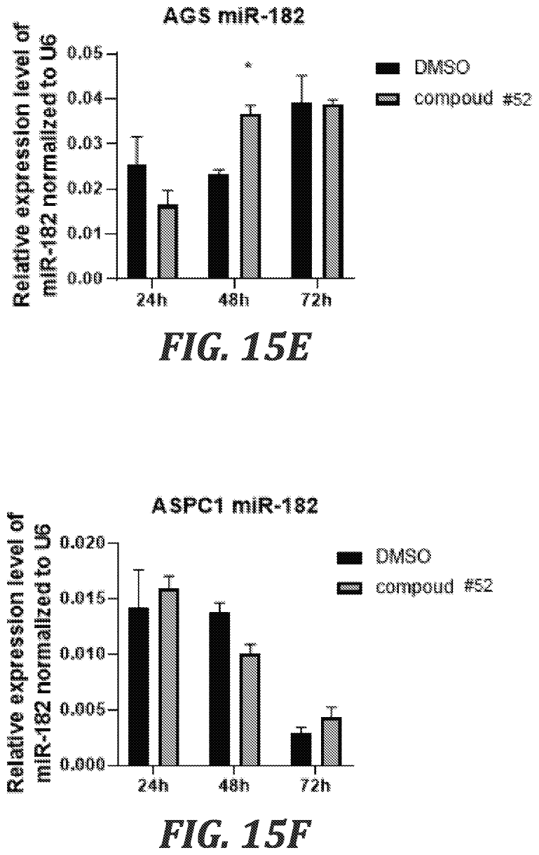

FIGS. 15A-15F show the levels of mature miR-16, miR-28, and miR-182 are not consistently affected by the compounds in both gastric (AGS) and pancreatic (ASPC1) cell lines, over multiple time scales (24-72 hours), relative to control where DMSO was added, but no compound.

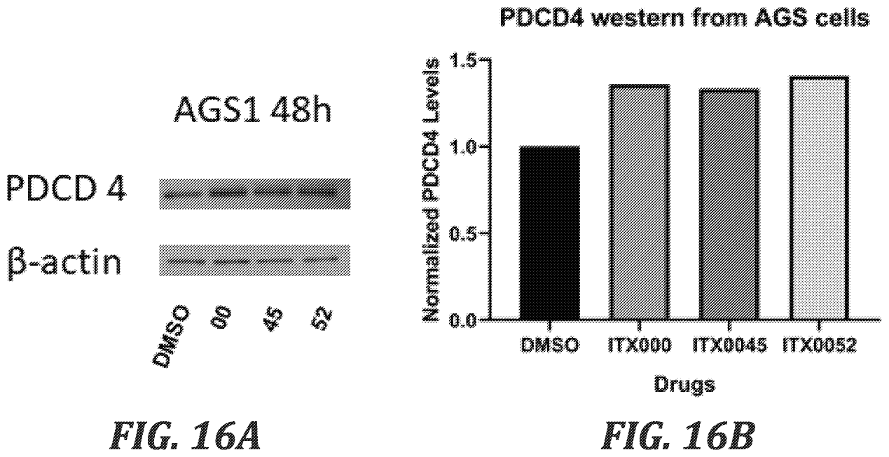

Finally, when levels of PDCD4 were measured, an increase was observed in the levels of the tumor suppressor induced by both −0045 and −0052 after 48 hours of incubation (FIGS. 16A and 16B), showing that the decrease in mature miR-21 levels also lead to an increase in at least the PDCD4 downstream target of miR-21.

Taken together, these data indicate that decreased cell proliferation for AGS and ASPC1 cells induced by −0052 occurs while the same compound also decreases levels of mature miR-21 and restores levels of the tumor suppressor PDCD4. Decreases in mature miR-10b are also observed under the same conditions, while other microRNAs are unaffected. Thus, inhibition of miR-21 processing by certain compounds represented in FIG. 5 is specific (miR-16, −28, and −182 are not affected) and not simply a result of reduce proliferation (palbociclib does not significantly affect miR-21 levels).

The following examples are provided for the purpose of illustrating, not limiting, the invention.

### EXAMPLES

Methods

RNA Transcription. All RNAs for ligand screening and structural or biochemical work were prepared in house using in vitro transcription on a large scale (typically 10 mL). RNA transcription and purification protocols used purified DNA oligonucleotide templates (IDT) and T7 RNA polymerase. Briefly, 1 mL of 8 μM TOP DNA (5′-CTATAGTGAGTCGTATTA-3′ (SEQ ID NO: 1)), corresponding to the phage T7 RNA polymerase promoter region, was annealed to 80 μL of 100 μM template sequences with 13 mM MgCl₂, heated to 95° C. for 4 mM then allowed to anneal to room temperature over 20 min. The annealing mixture was incubated with 5 mM of each of the four NTPs (ATP, GTP, UTP and CTP, from Sigma), transcription buffer, 8% PEG-8000 and 35 mM magnesium chloride with 0.4 mg/mL T7 RNA polymerase expressed and purified using established methods. All RNA samples are purified from crude transcriptions by 20% denaturing polyacrylamide gel electrophoresis (PAGE), electroeluted and concentrated by ethanol precipitation. The samples are re-dissolved in 12 mL of high salt wash (700 mM NaCl, 200 mM KCl, in 10 mM potassium phosphate at pH 6.5, with 10 μM EDTA to chelate any divalent ions), concentrated using Centriprep conical concentrators (3000 kDa MWC, Millipore). The RNA was then slowly exchanged into low salt storage buffer (10 mM potassium phosphate at pH 6.5, with 10 mM NaCl and 10 μM EDTA). Prior to NMR experiments, all RNA samples were finally desalted using NAP-10 gravity columns, lyophilized and re-dissolved in ‘screening buffer’ or ‘structure buffer’ (see below), then annealed by heating for 4 min to 90° C. and snap cooling at −20° C.

Compounds

Sources. Commercially available small molecule palboclib was purchased from Selleckchem. Starting materials were purchased from Abosyn Chemicals (compound −0052); or Combi-blocks, PharmaBlock, and Astatech.

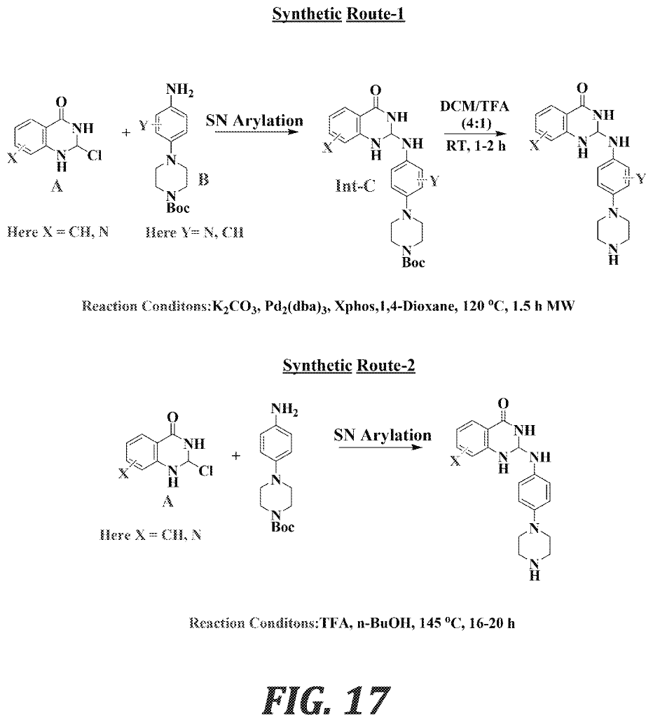

General Procedure: Route-1 (FIG. 17). The aniline (B, 1.05 equiv.) was taken up in dry 1,4-dioxane (0.1 M) in a microwave vial with a magnetic stir bar, 2-chloropyrimidine (A, 1.0 equiv.) was added followed by K₂CO₃ (3.0 equiv.) and X-Phos (0.2 equiv.). The reaction mixture (RM) was sparged with nitrogen gas for 5-10 minutes. Finally, Pd₂ (dba) 3 (0.1 equiv.) was added and RM was sparged with nitrogen for another 5 mM, the microwave vial was sealed and microwave irradiated at 120° C. for 1.5 h. The RM was cooled to ambient temperature, filtered through a pad of Celite, rinsed with ethyl acetate and the solvent was removed under reduced pressure. The crude RM was purified on silica gel using 0-80% ethyl acetate in hexanes as eluent. Relevant pure fractions were evaporated in vacuum to give the correspond intermediate-C. The corresponding Int-C were dissolved in DCM/TFA (4:1, 0.05 M), stirred for 1-2 hours at RT. The crude reaction was concentrated and purified on HPLC using water/acetonitrile as eluent. Relevant pure peak fractions were lyophilized to generate compound −0052 and analogs (overall yield for the two steps reaction: 20-50%).

General Procedure: Route 2 (FIG. 17). 1-Boc-4-(4-aminophenyl)piperazine (1.05 equiv.) and the corresponding 2-chloropyrimidine (A, 1.0 equiv.), TFA (3.0 equiv.) were taken in a sealed tube with n-BuOH (0.05 M). The reaction mixture (RM) was heated at 145° C. overnight (16-22 h). The RM was cooled down to ambient temperature and excess TFA was quenched with triethylamine (TEA). The crude compound was purified on HPLC using water/acetonitrile as eluent. Relevant pure peak fractions were lyophilized to give the corresponding −0052 analogs (yield 35%).

Biochemical Assays

Dicer/TRBP. The Dicer/TRBP enzyme complex was expressed and purified using published methods and stored at −80° C. in 20 μL aliquots until needed. Briefly, the gene encoding human full-length Dicer was inserted into a modified pFastBac1 vector (Invitrogen), which has an N-terminal glutathione-S-transferase (GST) tag. The GST fusion proteins were expressed in HiFive insect cells following standard procedures and isolated by glutathione affinity chromatography using buffer A (20 mM Tris-HCl pH 8.0, 150 mM NaCl, 2 mM DTT). After on-column cleavage by Tobacco Etch Virus (TEV) protease overnight, the proteins were eluted, concentrated and loaded onto Superdex 200 GL column equilibrated in buffer A. Sequences coding for human full-length TRBP2 were cloned into a modified pET28a vector with an N-terminal GB1 tag. The clones were transformed into Rosetta 2 cells and the transformants were grown in LB media at 37° C. until the cells reached a density corresponding to an OD reading of 0.8 at 600 nm. Protein expression was induced by addition of 0.2 mM IPTG at 18° C. for 20 hours. The cells were harvested, pelleted and resuspended in 20 mM Tris-HCl pH 8.0, 500 mM NaCl, mM imidazole, with 5 mM β-ME. Cells were lysed by sonication on ice then pelleted by centrifugation. The crude lysate was applied to a nickel affinity column and eluted in 20 mM Tris-HCl pH 8.0, 500 mM NaCl, 250 mM imidazole buffer, with 5 mM β-ME. TEV protease was added to remove the His-GB1 tag at 4° C. overnight. The sample was concentrated and loaded onto a Superdex 200 10/300 GL column equilibrated in buffer A. The purified Dicer and TRBP2 proteins were mixed at a molar ratio of 1:1.3 and loaded onto Superdex 200 10/300 GL column equilibrated in buffer A. The fractions containing the complex were pooled and concentrated to about 250 nM, then flash frozen for further use in processing assays.

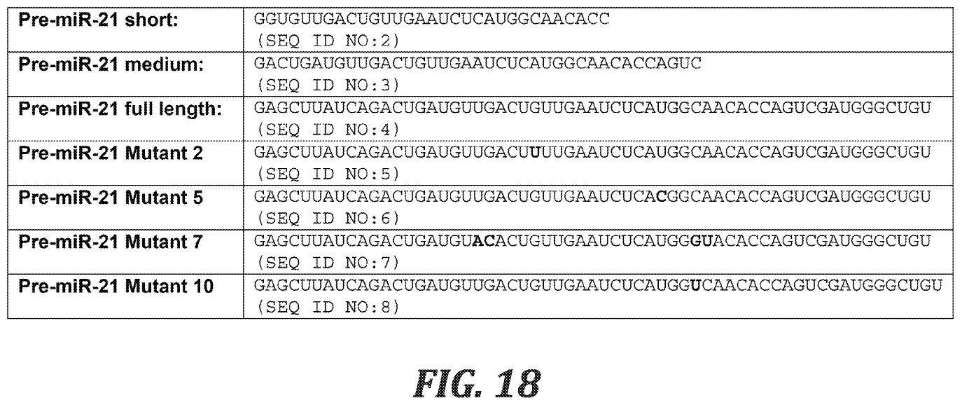

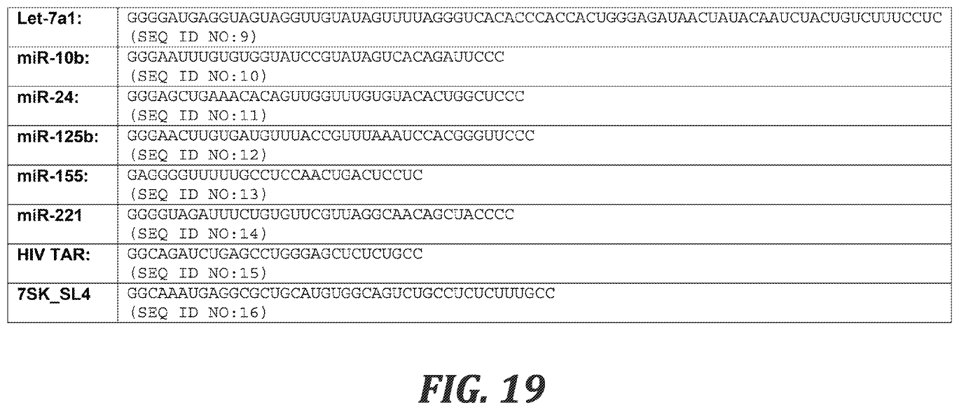

All pre-miRNA substrates were chemically synthesized by Integrated DNA Technologies (IDT) for efficient 5′-end labeling, which was done using a modified T4 PNK reaction; all sequences examined in this study are listed in FIGS. 18 and 19. Radiolabeling reactions were carried out in 30 μL volumes, containing 11 μL DNAse/RNAse free water, 3 μL 10× T4 PNK buffer, 3 μL of 10 μM pre-miRNA (IDT), 3 μL of 10 units/μL T4 PNK, 10 μL γ-³²P ATP (6000 Ci/mmol, 150 mCi/mL). Reactions proceeded for 1 hr at 37° C., followed by the addition of 50 mM EDTA to quench the reaction, along with heating to 95° C. for 10 min to inactivate the enzyme. After the PNK labeling reaction was completed, excess unincorporated nucleotides were removed using Zymo Research oligo clean up columns and RNA was eluted in 15 μL of Rnase/Dnase free water. An equal volume of 2× formamide gel loading buffer was added to the eluted RNA, followed by incubation at 95° C. for 5 min, then loaded onto 20% 19:1 denaturing (8 M urea) polyacrylamide gels. Gels were run for 1.25 hrs at 10 W, covered with plastic wrap and exposed to x-ray film for 30 sec to visualize the labeled RNA. The developed film was laid on the gel and oriented by internal phosphorescent paint positional controls placed near dye molecular weight markers. The area around the developed bands corresponding to the molecular weight of the target RNAs was excised and RNA was extracted from the gel slices by using Zymo Research ZR-small-RNA PAGE recovery kit, eluted into 15 μL of water and stored at −20° C. These fresh samples had a specific reactivity of 200 k CPM, as measured by a Geiger counter with an approximate RNA concentration of 2 μM.

Dicer/TRBP assays are run immediately (within 2 weeks) after preparation of the labeled RNA to maintain high levels of radioactivity, which are needed because miR-21 processing is inefficient and high sensitivity is required for accurate quantitation. Assays were conducted in 60 μL total volume with 25 nM RNA, 1 nM Dicer/TRBP in 1× reaction buffer (20 mM Tris at pH 7.5, 25 mM NaCl, 5 mM MgCl₂, 1 mM DTT and 1% glycerol). Assays were conducted in 96-well format in a PCR thermocycler in 0.2 mL PCR well plates to maintain constant temperature and improve reproducibility. This approach allows for direct comparison of enzyme and inhibitor activity for different pre-miRNA substrates (e.g., Let7, miR-21, mutant miRNAs) along with technical duplications to ensure data reproducibility with regards to enzyme aliquots. Each reaction well contained 25 μL of RNA substrate (typical stock RNA concentration was 67 nM but this concentration was titrated for kinetics work). Each enzyme well was filled with 50 μL of freshly diluted 2 nM Dicer-TRBP while each ligand well was filled with 50 μL of ligand at different concentrations (0-150 μM). Finally, 20 μL of reaction stop buffer (2× RNA load buffer, 95% formamide, 18 mM EDTA, 0.025% SDS, 0.1% xylene cyanol and 0.1% bromophenol blue) was added to the remaining wells.

Prior to initiating the reactions, 10 μL of ligand were added into the RNA wells using a multichannel pipette and allowed to equilibrate at 4° C. for 30 mM. The temperature of the thermocycler was then raised to 37° C. and samples were incubated for 15 mM to reach thermal equilibrium. Reactions were initiated by adding 25 μL of Dicer-TRBP to the RNA substrate wells (with or without small molecule ligand). The final enzyme concentration for all experiments was fixed to 1 nM while the final RNA and ligand concentrations were variable. The reactions were stopped by removing 5 μL from the reaction well and adding it to stop buffer wells at the indicated time points. Reactions were resolved by 20% 19:1 denaturing (8M urea) polyacrylamide gel and visualized using a Typhoon Gel imaging system (GE). Bands were quantified in ImageJ and results visualized as ratios of cleaved and uncleaved RNAs, after subtracting the background.

DGCR8 competition assay. Sequences coding for human DGCR8 constructs (amino acids 285-710 or 285-773) were cloned into a modified pET28a vector with an N-terminal GB1 tag. The clones were transformed into Rosetta 2 cells and the transformants were grown in LB media at 37° C. until reaching an OD₆₀₀=0.8. Protein expression was induced by addition of 0.2 mM IPTG at 18° C. for 20 hours. The cells were harvested, pelleted and resuspended in 20 mM Tris-HCl pH 8.0, 500 mM NaCl, 25 mM imidiazole, plus 5 mM β-ME. Cells were lysed by sonication on ice then pelleted by centrifugation. The crude lysate was applied to nickel affinity column and eluted by 20 mM Tris-HCl pH 8.0, 500 mM NaCl, 250 mM imidazole, 5 mM (3-mercapto-ethanol. TEV protease was added at 4° C. overnight to remove the His-GB1 tag. The proteins were diluted and purified further on a Resource S column equilibrated in 20 mM HEPES at pH 7.5, 250 mM NaCl, 1 mM DTT. After running a linear gradient against 20 mM HEPES pH 7.5, 1 M NaCl, 1 mM DTT, fractions containing the target proteins were concentrated and loaded onto Superdex 200 10/300 GL column equilibrated in the final buffer (20 mM HEPES pH7.5, 300 mM NaCl, 1 mM DTT and 5 μM Hemin). The proteins were pooled, concentrated to 120 μM and flash frozen for use in binding assay.

Binding assays between the DGCR8 protein and pre-miR-21 were carried out in 1× DGCR8 binding buffer (20 mM HEPES pH 7.5, 150 mM NaCl, 1 mM DTT, 5 μM Hemin and 3 mM MgCl₂) without or in the presence of tRNA and BSA competitors (same as 1× with added 10 μG/mL BSA, 20 μg/mL yeast tRNA). Binding reactions were conducted in 96 well format on a PCR thermocycler, per the Dicer/TRBP assays. Protein stocks were aliquoted to one row with 2-fold serial dilution into 1× DGRC8 buffer (no competitors) from a 2 μM stock of DGCR8 in one column, reduced to no protein in column 1 Each RNA occupied subsequent rows and was aliquoted as 10 μL in columns 1-12 of 2 nM RNA (5′-end labeled, see above) reconstituted in 1× DGCR8 binding buffer (with or without competitors). Binding assay were initiated by adding 10 μL of protein stock to the RNA wells with a multichannel pipette and allowed to equilibrate at 4° C. for 30 mM prior to resolving complexes on 6% (37.5:1) non-denaturing PAGE at 4° C. Because the typical dyes (bromophenol blue and xylene cyanol) can bind to RNA stem-loop, the gel loading buffer was 10 μL containing 30% glycerol but without any dye markers.

Cellular Experiments

Cell line maintenance. Using the Cancer Cell Line Encyclopedia (CCLE), in-house cancer cell lines were screened to identify cancer and tissue types with varying levels of miR-21 expression. Experiments were performed using the human-derived cell lines AGS (gastric cancer) and AsPC-1 (pancreatic cancer). Cell lines were obtained from the American Type Culture Collection and maintained per instructions from the supplier. AGS were cultured in F12K medium supplemented with 10% FBS. AsPC-1 were cultured in RPMI, with 10% FBS added. All cells were kept at 37° C. in a humidified atmosphere of 5% CO₂ and underwent regular mycoplasma testing. All experiments were conducted when cells were 70-80% confluent.

Proliferation assay. The impact of the compound on cellular proliferation was assessed, with 5,000 cells in each cell line seeded in replicates of 6 in a 96 well plate and treated with DMSO or with compounds. After 48 hours, cells were treated with the MTS reagent and optical density was read to generate dose-response curves.

Quantitative real time PCR analysis. RNA was isolated utilizing Trizol (Invitrogen) and the Direct-zol RNA Miniprep kit (Zymo Research). RNA concentration was assessed with the NanoDrop ND-1000 spectrophotometer (ThermoFisher Scientific). All microRNAs expression was tested in the two representative cell lines using qRT-PCR. Cells were seeded in 6-well plates 24 hours before treatment, at a cell confluency of 50-60%. Each cell line was treated with 10 μM compound or with only the dimethyl sulfoxide (DMSO) solvent. RNA was collected after the 48-hour time point. Expression of miR-21 was assessed utilizing the TaqMan microRNA assay (Applied Biosystems). The complementary DNA (cDNA) was synthesized using the TaqMan Reverse Transcription Reagents kit (Applied Biosystems) and was used with the TaqMan probes and Supermix (BioRad) for qRT-PCR analysis. U6 or U48 were used as internal controls for RNA levels. All experiments were performed in triplicate, with all samples normalized to the internal controls and relative expression levels calculated using the 2^{ΔΔCt} method.

Western blot analysis. Western blot analysis was conducted according to standard protocols. Briefly, proteins were collected from cells and lysed and the Bradford assay was used to measure protein concentrations. Quantification of protein expression was conducted using the image analysis software ImageJ.

While illustrative embodiments have been illustrated and described, it will be appreciated that various changes can be made therein without departing from the spirit and scope of the invention.

1. A compound having formula (I): 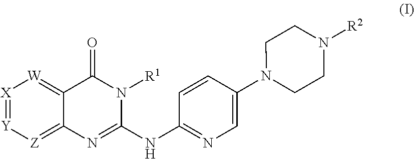
or a tautomer, or pharmaceutically acceptable salt thereof, wherein:
W, X, Y, and Z are independently selected from
(a) N, or,
(b) CR³;
R¹ is selected from the group consisting of hydrogen and unsubstituted C1-C6 alkyl;
R² is selected from the group consisting of hydrogen and unsubstituted C1-C6 alkyl; and
R³ is selected from the group consisting of hydrogen and unsubstituted C1-C6 alkyl,
with the proviso that only one of W, X, Y, and Z is N; or W is CR³, X is N, and Y is CR³; or W, X, Y, and Z are CR³.

2. The compound of claim 1 having formula (II): 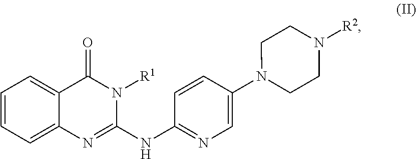
or a tautomer, or pharmaceutically acceptable salt thereof.

3. A compound of claim 1 having formula (III): 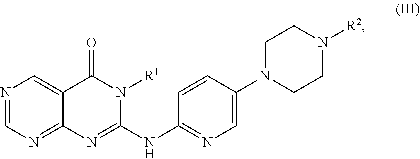
or a tautomer, or pharmaceutically acceptable salt thereof.

4. A compound of claim 1 having formulae (IVA), (IVB), (IVC), or (IVD): 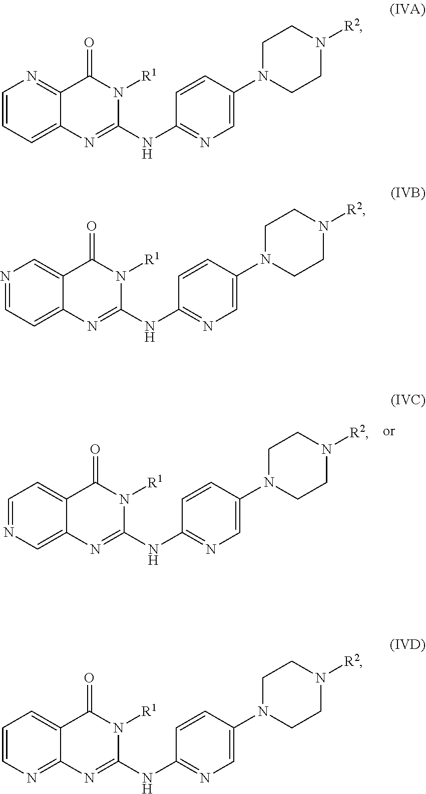
or a tautomer, or pharmaceutically acceptable salt thereof.

5. The compound of claim 2, or a tautomer, or pharmaceutically acceptable salt thereof, wherein R¹ is hydrogen and R² is hydrogen.

6. The compound of claim 2, or a tautomer, or pharmaceutically acceptable salt thereof, wherein R¹ is methyl and R² is hydrogen.

7. The compound of claim 3, or a tautomer, or pharmaceutically acceptable salt thereof, wherein R¹ is hydrogen and R² is hydrogen.

8. The compound of claim 4 having formula (IVA), or a tautomer, or pharmaceutically acceptable salt thereof, wherein R¹ is hydrogen and R² is hydrogen.

9. The compound of claim 4 having formula (IVB), or a tautomer, or pharmaceutically acceptable salt thereof, wherein R¹ is hydrogen and R² is hydrogen.

10. The compound of claim 4 having formula (IVC), or a tautomer, or pharmaceutically acceptable salt thereof, wherein R¹ is hydrogen and R² is hydrogen.

11. The compound of claim 4 having formula (IVD), or a tautomer, or pharmaceutically acceptable salt thereof, wherein R¹ is hydrogen and R² is hydrogen.

12. A pharmaceutical composition comprising a compound of claim 1, or a tautomer, or pharmaceutically acceptable salt thereof, and a pharmaceutically acceptable carrier.

13. A method for decreasing the level of microRNA miR-21 in a cell, comprising contacting a cell having upregulated miR-21 with an amount of a compound of claim 1, or a tautomer, or pharmaceutically acceptable salt thereof, effective to decrease the level of microRNA miR-21 in the cell.

14. (canceled)

15. A method for treating a disease or condition treatable by decreasing the level of microRNA miR-21 in a subject, comprising administering to a subject in need thereof a therapeutically effective amount of a compound of claim 1, or a tautomer, or pharmaceutically acceptable salt thereof.

16. The method of claim 15, wherein the disease or condition is proliferative disease or condition, irritable bowel disease, fibrosis of the kidney or liver, or a degenerative liver condition.

17. The method of claim 15, wherein the disease or condition is a cancer.

18. The method of claim 15, wherein the disease or condition is a cancer selected from glioma, glioblastoma, breast, ovarian, cervical, colon, non-small cell lung cancer, lung adenocarcinomas, gastric, pancreas, prostate, stomach, colorectal, liver, hepatoma and hepatocellular carcinomas, oral and tongue squamous cell carcinomas, head and neck, and larynx cancers.

19. The method of claim 15, wherein the disease or condition is a hematological malignancy.

20. The method of claim 15 further comprising administering to the subject a therapeutically effective amount of a second chemotherapeutic agent.

21. A method for inhibiting miR-21 in a subject, comprising administering to a subject in need thereof an amount of a compound of claim 1, or a tautomer, or pharmaceutically acceptable salt thereof, effective to inhibit miR-21 in the subject.
